(19) United States
(12) Patent Application Publication
(10) Pub. No.: US 2004/0134821 A1
(43) Pub. Date: Jul. 15, 2004

(54) Process for the sterile packaging of a prosthetic implant made of polyethylene

(76) Inventor: Alain Tornier, Saint Ismier (FR)

Correspondence Address: DOWELL & DOWELL PC, SUITE 309, 1215 JEFFERSON DAVIS HIGHWAY, ARLINGTON, VA, 22202

(21) Appl. No.: 10/730,276

(22) Filed: Dec. 9, 2003

(30) Foreign Application Priority Data
Dec. 10, 2002 (FR) 02 15600

(51) Int. Cl. A61L 15/00

(52) U.S. Cl. 206/438

(57) ABSTRACT

In order to package a prosthetic implant made of polyethylene in sterile manner, this implant is placed in a flexible, gas-impermeable sachet adapted to be closed, a vacuum is created in this sachet before it is hermetically closed, this sachet is placed in a gas-impermeable envelope adapted to be closed, an inert gaseous atmosphere is formed in the envelope, the envelope is closed hermetically, and the assembly formed by the implant, the sachet and the envelope is exposed to radiation.

The invention is more particularly applicable to the packaging of implants made of high density polyethylene.

### FIELD OF THE INVENTION

The present invention relates to a process for the sterile packaging of a prosthetic implant made of polyethylene.

The invention is particularly applicable to the packaging of high density polyethylene (HDPE) implants, particularly for knee or hip prostheses.

### BACKGROUND OF THE INVENTION

Between their manufacture and their implantation in a living being, such implants must be stored under good conditions of sterility, while allowing transport thereof. In order to sterilize these pieces which do not withstand high temperatures, it is known to use ionizing rays, particularly γ (gamma) rays. Moreover, in order to ensure that no subsequent contamination occurs, the implants are packed so as to be impermeable to the ambient air.

However, it is now known that, if polyethylene implants are exposed to radiation while the gaseous atmosphere surrounding the implants contains oxygen, phenomena of oxidation of the polyethylene occur. More precisely, the exposure to radiation provokes the break of polymeric chains of the polyethylene which, in the presence of oxygen, recombine with the latter, leading to the reduction of the molecular weight of the polyethylene and to the degradation of its mechanical properties. In the absence of oxygen, the polymeric chains recombine together, increasing the rate of cross-linking of the polymer, which guarantees good mechanical properties of the implant.

This is the reason why one type of process presently employed consists in firstly placing an implant in a flexible, gas-impermeable sachet, then in creating a vacuum in this sachet before closing it hermetically, and finally in sterilizing the implant contained in the sachet in vacuo by exposure to radiation.

Nonetheless, the use of such a sachet in vacuo remains delicate as it is difficult to guarantee the long-term tightness of the package, particularly during transport thereof, the least defect in closure of the sachet or the presence of a fragilized zone of the sachet compromising the sterile packaging of the implant.

It is an object of the present invention to propose a process of the afore-mentioned type, in which a polyethylene implant is sterilized satisfactorily while guaranteeing a long-term sterile environment of the implant, particularly during transport thereof.

### SUMMARY OF THE INVENTION

To that end, the invention relates to a process in which, successively, the implant is placed in a flexible, gas-impermeable sachet comprising an opening adapted to be sealed, a vacuum is created in the sachet before it is closed hermetically by sealing its opening, and the implant placed in the sachet in vacuo is sterilized by irradiation, characterized in that it comprises steps carried out successively before the irradiation of the implant placed in the first sachet in vacuo and consisting in:

placing the sachet in vacuo containing the implant in a gas-impermeable envelope comprising an opening adapted to be sealed,

forming an inert gaseous atmosphere in the envelope, and

closing the envelope hermetically by sealing its opening.

The packaging obtained by such a process guarantees that the ambient air, particularly the oxygen that it contains, cannot come into contact with the implant, even if the tightness of the sachet is compromised.

According to other characteristics of this process, taken separately or in any technically possible combinations:

closure of the sachet and/or of the envelope is effected by heat-sealing of their respective openings.

the inert gaseous atmosphere formed in the envelope is constituted by argon, nitrogen or a mixture of these gaseous elements.

the sachet comprises a layer of aluminum.

the envelope comprises a layer of polyamide and a layer of polyethylene.

in order to form the inert gaseous atmosphere in the envelope, the process comprises steps consisting in:

creating a vacuum around and inside the envelope, and

injecting an inert gas inside the envelope until the pressure inside the envelope reaches a predetermined value strictly less than atmospheric pressure,

and, after having hermetically closed the envelope, the latter is subjected to atmospheric pressure.

the inert gas is injected in calibrated manner.

before or after irradiation of the implant, the assembly formed by the implant, the sachet and the envelope is placed in a rigid packing whose internal volume is substantially equal to the volume occupied by the envelope.

before placing the assembly formed by the implant, the sachet and the envelope in the rigid packing, the envelope is folded on itself.

the rigid packing and the envelope cooperate by complementarity of shape in order to immobilize the sachet containing the implant.

### BRIEF DESCRIPTION OF THE DRAWINGS

The invention will be more readily understood on reading the following description given solely by way of example and made with reference to the accompanying drawings, in which:

FIG. 1 is a view in perspective of a packaging obtained by a process according to the invention.

FIG. 2 is a schematic view illustrating a first phase of the process carried out to obtain the packaging of FIG. 1.

FIG. 3 is a diagram showing the variation of pressure as a function of time within a sachet used in the first phase of the process illustrated in FIG. 2.

FIG. 4 is a view similar to FIG. 2, illustrating a second phase of the process carried out for obtaining the packaging of FIG. 1; and

FIG. 5 is a diagram showing the variation of pressure as a function of time within an envelope used in the second phase of the process illustrated in FIG. 4.

### DESCRIPTION OF PREFERRED EMBODIMENTS

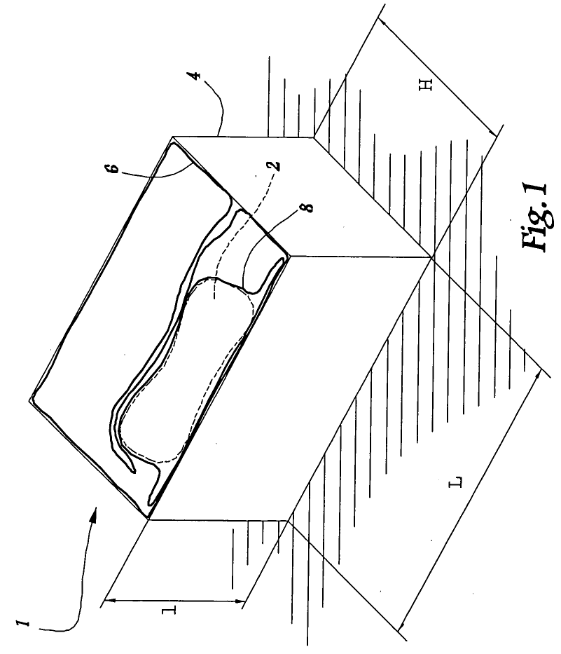

Referring now to the drawings, FIG. 1 shows a sterile packaging 1 for a prosthetic implant 2, comprising an outer packing 4, an outer envelope 6 and an inner sachet 8.

The implant 2 is for example an acetabulum made of high density polyethylene.

The outer packing 4 forms a rigid box of parallelepipedic shape, of dimensions L×1×H, as indicated in FIG. 1. This box is open on at least one of these faces. It is, for example, made of cardboard.

The outer envelope 6 presents a multi-layer structure and comprises at least one layer of polyamide and one layer of polyethylene, rendering it both flexible and gas-impermeable. Taking into account the conventional methods of manufacturing such an envelope, its impermeability is not necessarily strictly perfect.

The inner sachet 8 also presents a multi-layer structure and comprises at least one layer of aluminium and an inner layer of polyamide, rendering it both flexible, gas-impermeable and opaque to visible light.

Other characteristics of the outer envelope and of the inner sachet will appear from the following description of an example of process of packaging carried out in order to obtain the packaging 1. In the following specification, the pressures indicated are absolute pressures.

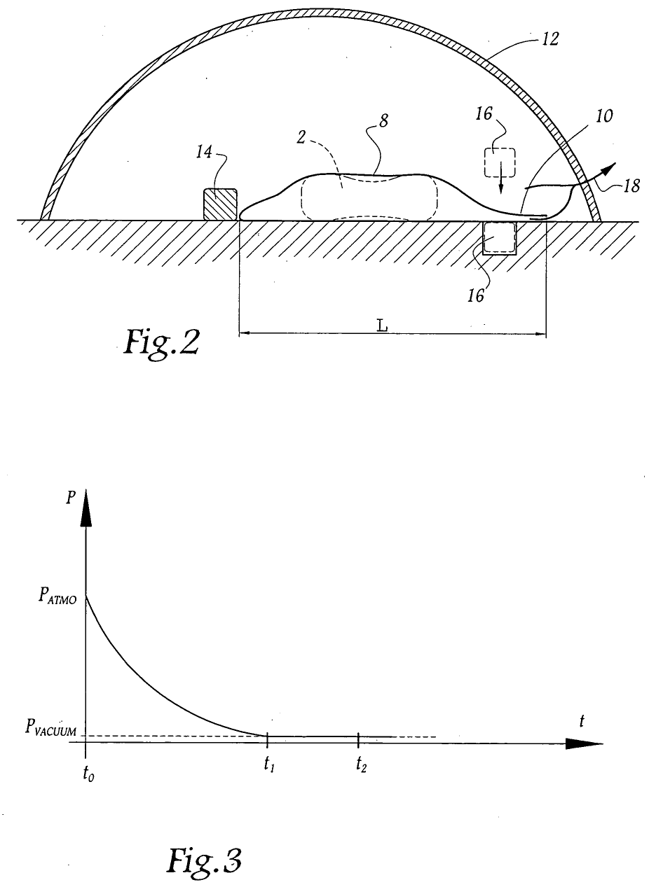

As shown in FIG. 2, the implant 2 is firstly placed in the inner sachet 8, of which the dimensions, flat, are advantageously a length of about L and a width of about 1. To that end, the sachet 8 comprises an opening 10 adapted to be sealed by fusion of the polyamide forming the inner layer of the sachet. The sachet containing the implant 2 is positioned beneath a bell 12, using a positioning bar 14 whose position is pre-established so that the opening 10 of the sachet is disposed between open heat-sealing jaws 16. The bell 12 is provided with vacuum-creating means (not shown).

More precisely, during a step represented between instants t₀ and t₀ in FIG. 3, the air initially contained in the bell 12 is evacuated therefrom, including that contained in the sachet 8, as symbolized by arrow 18 in FIG. 2, until the pressure prevailing in the sachet 8 attains a value of some millibars, denoted P_{VACUUM} in FIG. 3.

At instant t₁, the jaws 16 are then closed on themselves and, from t₁ to t₂, these jaws weld the edges of the opening 10 to each other, locally taking the polyamide forming the inner layer of the sachet to its melting temperature.

At instant t₂, the jaws are opened again and the chamber defined by the bell 12 is re-pressurized. The sachet 8 being hermetically closed, the pressure prevailing inside this sachet remains substantially equal to the pressure P_{VACUUM}. The quality of the weld may then be visually checked.

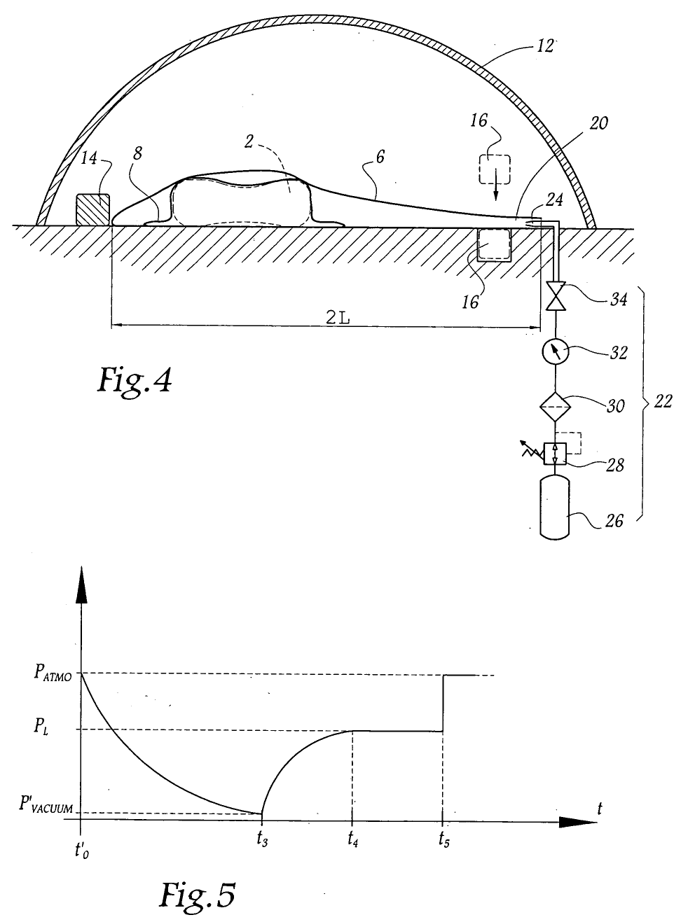

As shown in FIG. 4, the sachet 8 containing the implant 2 is then placed in the outer envelope 6 whose dimensions are advantageously a length equal to about 2×L and a width equal to about 1. To that end, the envelope 6 comprises an opening 20 adapted to be sealed by fusion of the polyamide which partly forms this sachet. The envelope is positioned in the bell 12, using the positioning bar 14 previously displaced with respect to its position of FIG. 2, so that the opening 20 is disposed between the open jaws 16.

In addition to the afore-mentioned vacuum-creating means, the bell 12 comprises argon-injecting means 22 intended to form an inert gaseous atmosphere within the envelope 6.

More precisely, during a step represented between instants t₀′ and t₃ in FIG. 5, the air initially contained in the bell 12, including that in the envelope 6, is evacuated until the pressure prevailing inside the sachet 8 attains a value of some millibars, denoted P′_{VACUUM} in FIG. 5. In order not to fragilize the inner sachet 8, care is taken that the value P′_{VACUUM} is equal to or slightly greater than the value P_{vacuum} of FIG. 3.

From t₃ to t₄, the injection means 22 are then employed so as to inject, via a nozzle 24 opening into the opening 20 of the envelope 6, argon coming from a bottle 26 storing argon at high pressure and passing successively from this bottle through a pressure reducing valve 28, a filtering member 30, a pressure gauge 32 and a control valve 34. The pressure gauge 32 ensures that the pressure of argon injected is of the order of 1 bar. The nozzle 24 is calibrated so that the flowrate of the argon is sufficiently low and stable to avoid blowing of the envelope.

This injection step continues until the pressure prevailing inside the envelope 6 attains a predetermined value, denoted P_{L} in FIG. 5, strictly less than atmospheric pressure, denoted P_{ATMO}. The pressure P_{L} is chosen between 0.3 and 0.7 bar. It is advantageously about 0.5 bar.

At instant t₄, the jaws 16 are closed on themselves and, from t₄ to t₅, they weld the edges of the opening 20 to each other.

At instant t₅, the jaws are opened again, the argon injection means 22 are stopped and the bell 12, after having possibly been re-pressurized further, is opened. The envelope 6 being hermetically closed, the gaseous atmosphere prevailing inside this envelope passes rapidly from pressure P_{L} to atmospheric pressure P_{ATMO} and the volume occupied by the envelope is reduced, by deformation in compression of the flexible multi-layer structure of the envelope.

The assembly formed by the implant 2, the envelope 6 and the sachet 8 is then placed inside the rigid packing 4, folding the envelope once on itself so that its space requirement in length is about L. The volume occupied by the envelope 6 is dimensioned so as to be inscribed in substantially complementary manner in the internal volume of the packing 4, with the result that the inner sachet 8 containing the implant is immobilized, as represented in FIG. 1.

In order to sterilize the implant 2, the packaging 1 formed by the implant, the envelope 6, the sachet 8 and the packing 4 is then exposed to γ (gamma) rays, possibly after having been transported up to a source of radiation.

All the packaging operations described hereinabove are carried out in a clean room.

The inert gaseous atmosphere formed by argon in the sterile packaging 1 thus obtained both ensures for the polyethylene implant a barrier against the ambient air, particularly the oxygen that it contains, in particular in the event of the tightness of the inner sachet being broken, and provides a function of immobilization ensuring shock absorption when the packaging is transported. The slight compression of the flexible outer envelope 6 when it is returned to atmospheric pressure tends to reinforce its tightness with respect to the ambient air, while cancelling the stresses of pressure between the interior and exterior of this envelope since the pressures prevailing on either side of the walls of the flexible envelope are equal.

Furthermore, the sterile packaging obtained is less expensive and occupies less space than a rigid packing in which an implant is mechanically immobilized, for example by shims of cellular material.

Various variants and arrangements of the process which has just been described may be envisaged:

apart from argon, the inert gaseous atmosphere of the outer envelope may be formed by nitrogen or a mixture of argon and nitrogen.

the inner sachet may be of the same nature as the outer envelope, i.e. comprising layers of polyamide and polyethylene.

the outer envelope may be formed by a rigid or semi-rigid shell.

the bell provided with the means for injecting the inert gas inside the outer envelope may be different from the one creating a vacuum in the inner sachet; and/or

the steps consisting in obtaining the inner sachet in vacuo on the one hand, and in obtaining the outer envelope with inert atmosphere on the other hand, may be successively carried out without returning the inner sachet to the open air, on condition that a bell provided with adequate means be available.

What is claimed is:

1. Process for the sterile packaging of a prosthetic implant made of polyethylene, of the type in which, successively, the implant is placed in a flexible, gas-impermeable sachet comprising an opening adapted to be sealed, a vacuum is created in the sachet before it is closed hermetically by sealing its opening, and the implant placed in the sachet in vacuo is sterilized by irradiation,
wherein it comprises steps carried out successively before the irradiation of the implant placed in the first sachet in vacuo which consist in:
placing the sachet in vacuo containing the implant in a gas-impermeable envelope comprising an opening adapted to be sealed,
forming an inert gaseous atmosphere in the envelope, and
closing the envelope hermetically by sealing its opening.

2. The process of claim 1, wherein the closure of the sachet and/or of the envelope is effected by heat-sealing their respective openings.

3. The process of claim 1, wherein the inert gaseous atmosphere formed in the envelope is constituted by argon, nitrogen or a mixture of these gaseous elements.

4. The process of claim 1, wherein the sachet comprises a layer of aluminum.

5. The process of claim 1, wherein the envelope comprises a layer of polyamide and a layer of polyethylene.

6. The process of claim 1, wherein it comprises, in order to form the inert gaseous atmosphere in the envelope, steps consisting in:
creating a vacuum around and inside the envelope, and
injecting an inert gas inside the envelope until the pressure inside the envelope reaches a predetermined value strictly less than atmospheric pressure,
and, after having hermetically closed the envelope, the latter is subjected to atmospheric pressure.

7. The process of claim 6, wherein the inert gas is injected in calibrated manner.

8. The process of claim 1, wherein, before or after irradiation of the implant, the assembly formed by the implant, the sachet and the envelope is placed in a rigid packing whose internal volume is substantially equal to the volume occupied by the envelope.

9. The process of claim 8, wherein, before placing the assembly formed by the implant, the sachet and the envelope in the rigid packing, the envelope is folded on itself.

10. The process of claim 8, wherein the rigid packing and the envelope cooperate by complementarity of shape in order to immobilize the sachet containing the implant.
